(19) United States
(12) Patent Application Publication
(10) Pub. No.: US 2012/0121613 A1
(43) Pub. Date: May 17, 2012

(54) PROTEIN CONJUGATE HAVING AN ENDOPEPTIDASE- CLEAVABLE BIOPROTECTIVE MOIETY

(75) Inventors: Liang Tang, Hercules, CA (US); Jun Wang, Nazaretto, PA (US); Baisong Mei, Waban, MA (US); John E. Murphy, Berkeley, CA (US)

(73) Assignee: BAYER HEALTHCARE LLC, Tarrytown, NY (US)

(21) Appl. No.: 13/145,335

(22) PCT Filed: Jan. 19, 2010
(86) PCT No.: PCT/US2010/021439

§ 371 Date: Feb. 1, 2012

Related U.S. Application Data
(60) Provisional application No. 61/145,644, filed on Jan. 19, 2009.

(51) Int. Cl. A61K 38/36; C12N 9/96; A61K 38/48; C07K 17/08; A61K 38/37; A61K 38/38; C07K 17/10; C07K 17/00; A61P 7/04

(52) U.S. Cl. 424/178.1; 530/381; 435/188; 424/94.3; 514/13.7; 514/14.1; 530/362; 530/383

(57) ABSTRACT

The invention is directed to a procoagulant conjugate having an endopeptidase-activatable procoagulant protein moiety and one or more bioprotective moieties, which are conjugated to one another by a linker that is cleaved by an endopeptidase in situ to release the bioprotective moiety. The invention is also directed to therapeutic uses of the procoagulant conjugate and methods of making the conjugate.

This application claims benefit of U.S. Provisional Application Ser. No. 61/145,644; filed on Jan. 19, 2009, the contents of which are incorporated herein by reference in their entirety.

### FIELD OF THE INVENTION

The invention relates in general to protein conjugates, to methods of making the protein conjugates, and to therapeutic use of the protein conjugates.

### BACKGROUND OF THE INVENTION

About one out of five thousand men worldwide suffer from bleeding disorders caused by lack of an effective amount of a coagulation factor (hemophilia). The most common hemophilia (type A) results from defects in a procoagulant factor, Factor VIII (FVIII), which lies in the intrinsic pathway of the blood coagulation cascade. A deficiency of functional FVIII results in a prolonged coagulation time and deleterious sequelae. Some hemophilic patients develop inhibitors (e.g., inhibitory antibodies) to administered FVIII. Type B hemophilia results from inadequate functional Factor IX (FIX).

Current treatment of patients having hemophilia A involves regular intravenous injection of recombinant coagulation FVIII (rFVIII), which is normally performed every 2 to 3 days due to the relatively short half-life of rFVIII in the blood stream. Other blood coagulation factors, such as FIX and activated Factor VII (FVIIa), have been used in the treatment of different hemophilia diseases. The limited half-life of blood coagulation factors in vivo, requires frequent infusion of therapeutic factors into hemophilia patients. Thus, hemophilia B patients benefit from 2-3 infusions of FIX/week. Therapy by multiple administration of purified FVIIa per day is advantageous for overcoming FVIII deficiency in patients who elaborate inhibitors of FVIII.

Attaching a bioprotective moiety or multiple bioprotective moieties (e.g., addition of a polyethylene glycol moiety, or PEGylation) to coagulation factors can increase the half-life of those molecules in vivo and can improve the treatment of diseases related to the deficiency of blood coagulation factors in patients (see, e.g., US Patent Application Nos. 2008/0039373, 2006/0252689, and 2008/0102115). Moreover, antibody development can be inhibited, protease digestion can be attenuated, and removal by kidney filtration can be slowed by PEGylation (Harris, et al., Clinical Pharmacokinetics 40:539-551, 2001). PEGylation may also increase the overall stability and solubility of the protein. The sustained plasma concentration of PEGylated therapeutic proteins can reduce the extent of adverse clinical effects by reducing the trough to peak levels of the therapeutic protein, thus moderating the need to introduce super-physiological levels of the protein.

FVIII having a bioprotective moiety conjugated to one or more of several sites on FVIII has been reported (see, e.g., WO 94/15625; U.S. Pat. No. 4,970,300; U.S. Pat. No. 6,048,720; US Patent Application Publication No. 2006/0115876). For example, site-directed mutation of the nucleotide sequence may be used to introduce a reactive amino acid residue at the surface of FVIII wherein the introduced amino acid residue(s) is a point of attachment for a PEG moiety.

As an alternative to direct reaction of a bioprotective group onto a protein, use of a linker or spacer can be advantageous as a means to space the bioprotective group at a distance from the protein (see, e.g., WO 2007/140282 directed to bifunctional PEG linkers; WO 2005/112919 directed to self-immolative peptidyl spacers).

Thus, the skilled artisan will appreciate that the introduction of a bioprotective moiety such as a hydrophilic polymer (e.g., a PEG moiety) onto a therapeutic protein can be advantageous in certain respects, but can negatively impact the activity profile of the protein in vivo. It is now possible to provide a conjugate wherein the bioprotective moiety is released from the protein at the in vivo microlocus where protein activity is needed.

### SUMMARY OF THE INVENTION

The present invention provides a modified procoagulant factor which exhibits prolonged half life in blood (and other advantages associated with its conjugation to a bioprotective moiety) while providing unencumbered or less encumbered activity at a targeted in vivo microlocus, that is, a bleeding site where fibrinogen is converted proteolytically to fibrin.

In one embodiment, the invention comprises a procoagulant factor conjugate comprising an endopeptidase-activatable procoagulant factor and at least one bioprotective moiety linked thereto by at least one linker, wherein the linker comprises at least one cleavage site recognized by an endopeptidase which activates the endopeptidase-activatable procoagulant factor, such that the bioprotective moiety is substantially released in the presence of the endopeptidase. The invention also comprises a therapeutic composition comprising such a conjugate.

In another embodiment, the invention comprises a method of making a procoagulant factor conjugate comprising an endopeptidase-activatable protein and a bioprotective moiety linked to the procoagulant factor, the method comprising covalently coupling said bioprotective moiety to a linker comprising a polypeptide having at least one cleavage site for an endopeptidase and a covalently bound reactive group, and reacting said reactive group with the procoagulant factor to covalently attach the linker bearing the bioprotective moiety to the procoagulant factor.

The invention also comprises a method of treating a coagulation factor deficiency in a subject, which comprises administering to the subject a therapeutically-effective amount of an endopeptidase-activatable procoagulant factor conjugate, which conjugate comprises a procoagulant factor moiety conjugated to a bioprotective moiety by means of a linker, wherein the linker provides at least one cleavage site which is recognized by an endopeptidase, whereby the linker is cleaved in vivo to provide substantially unconjugated procoagulant factor. In one aspect of this method, the endopeptidase is thrombin and the cleavage site is a cleavage site recognized by thrombin. In another aspect, the coagulant factor may be FV, FVII, FVIII, FIX, FX, and thrombin or a procoagulant factor thereof.

In another embodiment, the invention comprises a FVIII conjugate which is reactive in vivo to provide FVIII or FVIIIa at a site of proteolytic conversion of fibrinogen to fibrin, which conjugate comprises a FVIII moiety and a bioprotective moiety, which conjugate is cleaved in vivo at the site of proteolytic conversion to provide substantially unconjugated FVIII at the site of proteolytic conversion.

### DESCRIPTION OF THE FIGURES

FIG. 1 depicts the domain structure of a FVIII-linker-PEG conjugate wherein a FVIII moiety is conjugated to a PEG moiety, and wherein the linker comprises a cleavable peptide which is either joined directly to FVIII or optionally has an intervening short-chain PEG spacer.

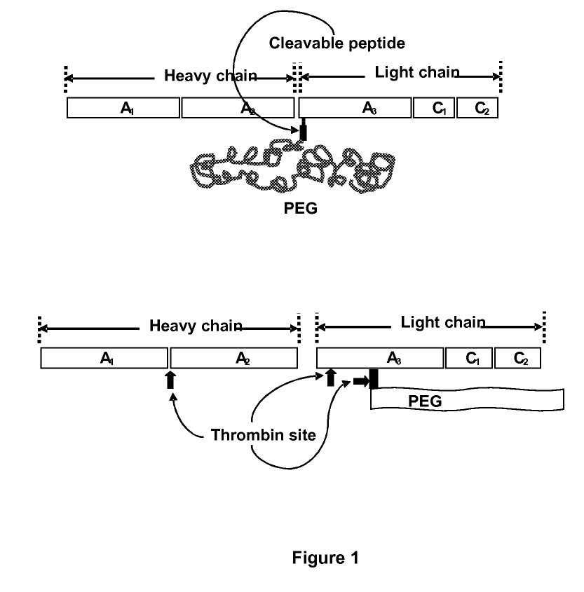

FIG. 2 depicts purification of a linker-PEG by Sephadex G-75 chromatography. The linker-PEG is prepared by reaction of PEG-butyraldehyde with the N-terminal amine of a short polypeptide followed by reduction of the resultant Schiff base. Panel A shows resolution of peptide-PEG from peptide by HPLC. Panel B shows iodine-stained gel lanes of the mixture before purification and the isolated peptide-PEG.

FIG. 3 depicts molecular weight analysis of a rFVIII mutein, the mutein after reaction with linker-PEG, and an analysis of the reaction. Panel A shows Coomassie Brilliant Blue- and Iodine-stained gels before and after purification by C7F7 immunoprecipitation. Panel B shows chromatography of immuno-purified reaction mixture on Superdex-200.

FIG. 4 depicts separation of PEG from PEGylated rFVIII following thrombin cleavage. Panel A shows a polyacrylamide electrophoresis gel having lanes 1) unmodified rFVIII mutein, 2) unmodified rFVIII mutein after treatment with thrombin, 3) PEG-modified rFVIII mutein, 4) PEG-modified rFVIII mutein after treatment with thrombin, 5) PEG-linker-modified rFVIII mutein, 6) PEG-linker-modified rFVIII mutein after thrombin treatment, and 7) thrombin. The gel is silver stained for protein. Panel B diagramatically depicts thrombin cleavage sites in the PEG-linker-modified rFVIII mutein. The gel is iodine stained for PEG

FIG. 5 is a Western blot which depicts the time course of removal of a PEG moiety from the light chain of FVIII by cleavage of a linker with thrombin.

### DESCRIPTION OF THE INVENTION

The term “endopeptidase activatable” means that an activity or proactivity of a protein (e.g., a coagulation factor) is increased upon hydrolysis of a peptide bond in the protein by the action of the endopeptidase. In one aspect, endopeptidase activation describes the conversion of a profactor to an active factor.

The “bioprotective moiety” is a chemical moiety which, when conjugated to a therapeutic protein, improves at least one activity of the protein when the protein is administered in vivo. The bioprotective moiety is selected from the group consisting of hydrophilic polymers including polyalkylene oxides, dextrans, polycarbohydrates including polysialic acids such as colominic acids, oligo- and poly-peptides, biotin derivatives, polyvinyl alcohol, polycarboxylates, polyvinylpyrrolidone, polyethylene-co-maleic acid anhydride, polystyrene-co-malic acid anhydride, polyoxazoline, polyacryloylmorpholine, heparin, celluloses, hydrolysates of chitosan, starches such as hydroxyethyl-starches and hydroxy propyl-starches, glycogen, agaroses and derivatives thereof, guar gum, pullulan, inulin, xanthan gum, carrageenan, pectin, alginate and alginic acid hydrolysates, albumin, immunoglobulin, and fragment of immunoglobulin, and combinations thereof. Bioprotective moieties can be branched, forked, multi-armed, or super-branched. A bioprotective moiety may be one or more hydrophilic polymers, polysaccharides for example starch, polysialic acid, albumin, immunoglobulin, or fragment of immunoglobulin, and combinations thereof. An exemplary hydrophilic polymer may be polyalkylene glycol. A hydrophilic polymer may be polyethylene glycol (PEG) or methoxypolyethylene glycol (mPEG). Other useful polyalkylene glycol compounds are polypropylene glycols, polybutylene glycols, PEG-glycidyl ethers, and PEG-oxycarbonylimidazole.

Typically, PEGs may comprise the following structure “—(OCH₂CH₂)ₙ—” where n is 2 to about 7000 or, for example, between about 70 and about 4000. As used herein, PEG also includes “—CH₂CH₂—O(CH₂CH₂O)ₙ—CH₂CH₂—” and “—(OCH₂CH₂)ₙO—,” depending upon whether or not the terminal oxygens have been displaced. Throughout the specification and claims, it should be remembered that the term “PEG” includes structures having various terminal or “end capping” groups, such as without limitation a hydroxyl or a C₁₋₂₀ alkoxy group. In context, the term “PEG” can also mean a polymer that contains greater than 50% of —OCH₂CH₂— repeating subunits. With respect to specific forms, the PEG can take any number of a variety of molecular weights, as well as structures or geometries such as branched, linear, forked, and multifunctional.

The bioprotective moiety used in the conjugates of the invention may be any of the polymers discussed above and protects the protein from one or more of loss of activity or potential activity, degradation, denaturation, inappropriate sequestration, or inappropriate proteolysis. The bioprotective moiety is selected to provide the desired improvement in pharmacokinetics. For example, the identity, size and structure of the bioprotective moiety is selected or empirically determined so as to improve or optimize the in vivo properties of the coagulation factor, and optionally to additionally decrease the antigenicity of the procoagulant factor, without an unacceptable decrease in activity. The bioprotective moiety may comprises PEG. In one embodiment, the polymer is a polyethylene glycol terminally capped with an end-capping moiety such as hydroxyl, alkoxy, substituted alkoxy, alkenoxy, substituted alkenoxy, alkynoxy, substituted alkynoxy, aryloxy and substituted aryloxy. In another embodiment, the polymers may comprise methoxypolyethylene glycol. The PEG may be between about 5 kD and about 300 kD or larger in size. In one embodiment, the PEG moiety is about 10 kD, about 20 kD, about 30 kD, about 40 kD, about 50 kD, about 60 kD, about 70 kD, about 80 kD, about 100 kD, about 120 kD, about 140 kD, about 160 kD, about 180 kD, about 200 kD, about 220 kD, about 240 kD, about 260 kD, about 280 kD, or about 300 kD or larger. Moreover, the PEG can be between about 10 kD and about 80 kD, between about 20 kD and about 70 kD, between about 30 kD and about 60 kD, between about 40 kD and about 80 kD, or between about 10 kD and about 40 kD.

The bioprotective moiety may be covalently coupled to a linker which is an oligo- or poly-peptide having one or more endopeptidase recognition and cleavage sites and a reactive moiety. The reactive moiety may comprise a moiety which can conjugate the bioprotective moiety to the procoagulation factor by any means of chemical conjugation, several of which means are conventional in the art. For example, in one embodiment, the linker is provided with a sulfhydryl reactive moiety which is reactive with a free cysteine (e.g., a cysteine introduced by mutation) on a coagulation factor to form a covalent linkage therebetween. Such sulfhydryl reactive moieties include thiol, triflate, tresylate, aziridine, oxirane, S-pyridyl, maleimidobenzoyl sulfosuccinimide ester, or maleimide moieties. In one embodiment, the bioprotective moiety and the linker are linear, the bioprotective moiety is attached to the linker and has a “cap,” such as methoxy, at one terminus that is not strongly reactive towards sulfhydryls, and the linker has a sulfhydryl reactive moiety at the other terminus. The polypeptide portion of the linker may comprise at least one endopeptidase cleavage site that is recognized by the same endopeptidase that activates the procoagulant factor. Thus, the linker can be cleaved in the presence of the said endopeptidase to release the protein (e.g., FVIII moiety) from the PEG moiety. It is understood that in the cleavage process, residual elements of the linker may remain with the FVIII, provided that these minor elements do not materially affect the activity of the procoagulant factor.

The linker may be a short, generally flexible molecular moiety that comprises amino acid residues forming at least one endopeptidase cleavage site and can also, but not necessarily, comprise a spacer consisting of a hydrophilic polymer segment, a nucleotide, an oligonucleotide, a saccharide, and/or an oligosaccharide. The linker can have up to about 50 amino acid residues. In one embodiment, the linker can have up to about 40 amino acid residues. In another embodiment the linker comprises up to about 30 amino acid residues. In yet another embodiment, the linker comprises up to about 20 amino acid residues. The linker may have a mass of less than about 5 kD, less than about 4 kD, or less than about 3 kD, and can be less than about 2 kD. A molecule used to form the linker is a “prolinker.” A prolinker typically has a reactive moiety that is capable of reacting with a protein.

The term “conjugate” means a covalent adduct of a procoagulant factor moiety and a bioprotective moiety, which includes a linker. The procoagulant factor can comprise a single polypeptide chain, two, three, or more polypeptide chains and can be a glycoprotein.

A “procoagulant factor” is a blood clotting factor zymogen which can be activated to form a clotting factor serine protease or a pro-cofactor which is needed for a clotting factor activity. The term procoagulant factor includes, for example, thrombin, FV, Factor VII (FVII), FVIII, FIX, and FX, which can be activated to form FVa, FVIIa, FVIIIa, FIXa, and FXa, respectively. The procoagulant factor may be human and may be recombinant. Muteins of the procoagulant protein which possess at least 10% of the procoagulant activity of the wild type factor are included.

A “FVIII moiety” is defined herein as FVIII protein which exhibits at least some of the biological activity of native FVIII. The FVIII moiety may be a human FVIII and may be recombinant. Included are muteins of Factor VIII which possess at least 10% and/or substantially all of the procoagulant activity of native FVIII. FVIII includes recombinant FVIII and FVIII having sequence deletions and muteins having sequence deletions, for example B-domain deleted FVIII, which lacks all or part of the B domain. A FVIII moiety has FVIII activity and may exist as part of a larger protein conjugate or may have additional chemical elements such as reactive moieties or residues thereof, oligopeptides, and short chain hydrophilic polymers.

An exemplary FVIII moiety is a BDD FVIII which is characterized by having the amino acid sequence which contains a deletion of all but 14 amino acids of the B-domain of FVIII. The first four amino acids of the B-domain are linked to the ten last residues of the B-domain. (Lind, et al., Eur. J. Biochem. 232:19-27, 1995).

FVIII is a glycoprotein synthesized and released into the bloodstream. In the circulating blood, it is bound to von Willebrand factor (vWF, also known as FVIII-related antigen) to form a stable complex. Upon activation by thrombin, FVIII is cleaved to form FVIIIa and dissociates from the complex to interact with other clotting factors in the coagulation cascade, which eventually leads to the formation of a thrombus.

As used herein, “functional FVIII polypeptide” denotes a functional polypeptide or combination of polypeptides that are capable, in vivo or in vitro, of correcting human FVIII deficiencies, reflected in the symptoms of hemophilia A. FVIII has multiple degradation or processed forms in the natural state. These are proteolytically derived from a precursor. A functional FVIII polypeptide includes such single chain protein and also provides for these various degradation products that have the biological activity of correcting, or mitigating the effects of, human FVIII deficiencies. Allelic variations likely exist. The functional FVIII polypeptides include all such allelic variations, glycosylated versions, modifications and fragments resulting in derivatives of FVIII in whole or in part, which characterizes native FVIII. The functional activity of derivatives of FVIII can readily be assessed by in vitro tests which are familiar to persons in the art and/or are described herein, for example, the COATEST assay. Furthermore, activated functional FVIII is a cofactor for catalyzing the conversion of Factor X to Xa in the presence of Factor IXa, calcium, and phospholipid. The fragments that can be derived via restriction enzyme cutting of the DNA or proteolytic or other degradation of human FVIII protein will be apparent to those skilled in the art.

The term Factor V moiety is used to mean human procoagulant Factor V and muteins that have at least 10% of the procoagulant activity of native Factor V. For example, the Factor V moiety may have substantially all of the procoagulant activity of native Factor V.

By a FVII moiety is meant human procoagulant Factor VII and muteins that have at least 10% of the procoagulant activity of native FVII. For example, the FVII moiety may have substantially all of the procoagulant activity of native FVII.

As used herein, a FIX moiety is human Procoagulant Factor IX and muteins thereof that have at least 10% of the procoagulant activity of native FIX. For example, the FIX moiety may have substantially all of the procoagulant activity of native FIX. FIX is also known as Human Clotting Factor IX and Plasma Thromboplastin Component.

As used herein, a FX moiety means human Coagulation Factor X, and muteins thereof that have at least 10% of the procoagulant activity of FX. For example, the FX moiety may have substantially all of the procoagulant activity of native FX.

A mutein is a genetically engineered protein arising as a result of a laboratory induced mutation to a nucleic acid which encodes the said protein.

The term “cleavage site” means an amino acid sequence that is recognized by an endopeptidase as a binding and hydrolytic site and encompasses the peptide bond that the endopeptidase hydrolyzes.

In the context of release of a bioprotective moiety from a population of procoagulant factor conjugates, substantial release means release of a bioprotective moiety in a very large proportion of such a population of protein conjugates (e.g., more than 50%). In the context of release of a bioprotective moiety from an individual or model conjugate, substantial release means that a very large proportion of the bioprotective moiety (e.g., more than 50%), or, for example, substantially all of the bioprotective moiety, is no longer covalently attached to the procoagulant factor moiety.

The endopeptidase-activatable procoagulant factor conjugate can have a protein or glycoprotein procoagulant factor moiety, a linker having an endoprotease cleavage site, and a bioprotective moiety.

In one embodiment, the protein/glycoprotein procoagulant moiety can be any blood procoagulant factor that is activatable by an endopeptidase. The procoagulant factor may be selected from the group consisting of FV, FVII, FVIII, FIX, FX, thrombin, and a mutein of any of those factors. As an example, the mutein may comprise up to about twenty amino acid substitutions, additions, and/or deletions. In one embodiment, the mutein may comprise up to about ten amino acid substitutions, additions, or deletions. In another embodiment, the mutein may comprise up to about six amino acid substitutions, additions, or deletions. In an additional embodiment, the mutein may comprise up to about three amino acid substitutions. The mutein can comprise one or several amino acid substitutions that introduce, for example, cysteine residues. In addition to the above, muteins can have deletions of non-functional or marginally functional protein sequences or domains. Exemplary embodiments having more extensive deletions are BDD FVIII and muteins thereof.

The endopeptidase-activatable protein conjugate can be activated by endopeptidase in vivo to provide an activated factor that has an activity substantially similar to a corresponding activity of a native factor.

In one embodiment, the endopeptidase is thrombin or Factor Xa.

In addition to the cleavage site, the linker can have one or more amino acid residues suitable for forming a coil, for example, G, S, and/or one or more of the sequences GG, GS, SG, and SS.

The linker may have at least one cleavage site which can be any endopeptidase cleavage site, for example, a thrombin cleavage site or a Factor Xa cleavage site. In one embodiment, the cleavage site may be selected from the group consisting of Xaa-Xab-Pro-Arg-Xac-Xad (SEQ ID NO. 1) and Gly-Arg-Xac-Xad (SEQ ID NO. 2), wherein Xaa and Xab are hydrophobic amino acid residues and Xac and Xad are amino acid residues other than acidic amino acid residues. In another embodiment, the cleavage site may be selected from the group consisting of Ile-Glu-Gly-Arg-Xaa (SEQ ID NO. 3), Ile-Asp-Gly-Arg-Xaa (SEQ ID NO. 4), and Ala-Glu-Gly-Arg-Xaa (SEQ ID NO. 5), wherein Xaa is an amino acid residue other than Arg or Pro. In another embodiment, Xaa is Ile or Thr. Thus, the sequences can be Ile-Glu-Gly-Arg-Ile/Thr (SEQ ID NO. 6), Ile-Asp-Gly-Arg-Ile/Thr (SEQ ID NO. 7), and Ala-Glu-Gly-Arg-Ile/Thr (SEQ ID NO. 8).

Potential sites for attachment of a linker having a bioprotective moiety, or for mutation in preparation for attachment of a linker having a bioprotective moiety, may be at or near the surface of the procoagulant factor and are accessible to the endopeptidase. The suitability of a particular attachment site can be assessed using an in vitro cleavage assay, an example of which is described in connection with thrombin in the examples which follow. For FIX, potential sites include any one or more of positions 39, 45, 51, 68, 89, 109, 127, 137, 146, 168, 189, 234, 247, 260, 274, 293, 311, 339, 347, 362, 387, 438, 440, 446, 455, 457, and 459, which are lysine residues in human FIX (see, e.g., US Patent Application No. 2006/00052302). Positions having outer surface aspartate, glutamate, or cysteine residues are also suitable. Surface lysine residues of FVII or FVIIa (see, e.g., US Patent Application No. 2007/0254840) and von Willebrand Factor (see, e.g., US Patent Application No. 2006/0160948) are also suitable for modification. Cysteines in von Willebrand Factor can also be modified.

The procoagulant factor contains at least one linked bioprotective moiety. Optionally, the procoagulant factor may have more than one cleavable, linked bioprotective moiety attached thereto, each attached by an individual linker. Optionally, the procoagulant factor may also comprise one or more conventionally-linked, non-endopeptidase cleavable bioprotective moieties attached thereto.

The conjugates and methods of the invention are described in still further detail in connection with an embodiment wherein a FVIII moiety is conjugated to a PEG moiety by a cleavable linker.

Site-directed mutation of a nucleotide sequence encoding polypeptide having a therapeutic function, for example FVIII activity, may be accomplished by any method known in the art. Methods include mutagenesis to introduce a cysteine codon at the site chosen for covalent attachment of the linker, which can be accomplished by known methods, such as the Stratagene cQuickChange™ II site-directed mutagenesis kit, the Clontech Transformer site-directed mutagenesis kit, the Invitrogen GenTaylor site-directed mutagenesis system, the Promega Altered Sites II in vitro mutagenesis system kit, or the Takara Mirus Bio LA PCR mutagenesis kit.

The conjugates of the invention may be prepared, for example, by first replacing the codon for one or more amino acids on the surface of the functional FVIII polypeptide with a codon for cysteine, producing the cysteine mutein in a recombinant expression system, reacting the mutein with a cysteine-specific linker reagent, and purifying the mutein.

The addition of a linker at the cysteine site can be accomplished through a maleimide-active functionality on the linker. The amount of sulfhydryl reactive polymer used should be at least equimolar to the molar amount of cysteines to be derivatized and preferably is present in excess. For example, at least a 5-fold molar excess of sulfhydryl reactive polymer is used, or at least a ten-fold excess of such polymer may be used. Specific conditions useful for covalent attachment are within the skill of those in the art.

The convention for naming muteins is based on the amino acid sequence for the mature, full length protein. Because secreted proteins contain a signal sequence that is proteolytically cleaved during the translation process, the sequence of the mature protein generally does not include the signal sequence. Following removal of the signal sequence from human FVIII, the first amino acid of the mature FVIII is alanine.

When referring to mutated amino acids in procoagulants such as BDD FVIII, the mutated amino acid is designated by its position in the sequence of the full-length procoagulant.

A predefined site for covalent binding of the linker having a bioprotective moiety may be selected from sites exposed on the surface of the polypeptide that are not involved in FVIII activity or involved in other mechanisms that stabilize FVIII in vivo, such as binding to vWF. Such sites may be selected from those sites known to be involved in mechanisms by which FVIII is deactivated or cleared from circulation. Sites may include an amino acid residue in or near a binding site for (a) low density lipoprotein receptor related protein, (b) a heparin sulphate proteoglycan, (c) low density lipoprotein receptor and/or (d) FVIII inhibitory antibodies. By “in or near a binding site” is meant a residue that is sufficiently close to a binding site such that covalent attachment of a biocompatible polymer to the site would result in steric hindrance of access to the binding site. Such a site is expected to be within 20 Å of a binding site.

In one embodiment, the linker having a bioprotective moiety may be covalently attached to the FVIII moiety at one or more of the FVIII amino acid positions 81, 129, 377, 378, 422, 468, 487, 491, 496, 504, 523, 556, 570, 711, 1648, 1795, 1796, 1803, 1804, 1808, 1810, 1812, 1813, 1815, 1864, 1903, 1911, 2091, 2118, and 2284. In another embodiment, the linker having a bioprotective moiety may be covalently attached to the FVIII moiety at one or more of FVIII amino acid positions 377, 378, 468, 491, 504, 556, 1795, 1796, 1803, 1804, 1808, 1810, 1864, 1903, 1911 and 2284 and (1) the binding of the conjugate to low-density lipoprotein receptor related protein is less than the binding of the unconjugated polypeptide to the low-density lipoprotein receptor related protein; (2) the binding of the conjugate to low-density lipoprotein receptor is less than the binding of the unconjugated polypeptide to the low-density lipoprotein receptor; or (3) the binding of the conjugate to both low-density lipoprotein receptor related protein and low-density lipoprotein receptor is less than the binding of the unconjugated polypeptide to the low-density lipoprotein receptor related protein and the low-density lipoprotein receptor.

In a further embodiment, the linker having a bioprotective moiety may be covalently attached to the polypeptide at one or more of FVIII amino acid positions 377, 378, 468, 491, 504, 556, and 711 and the binding of the conjugate to heparin sulphate proteoglycan is less than the binding of the unconjugated polypeptide to heparin sulphate proteoglycan. In a further embodiment, the bioprotective moiety may be covalently attached to the polypeptide at one or more of the FVIII amino acid positions 81, 129, 377, 378, 468, 487, 491, 504, 556, 570, 711, 1648, 1795, 1796, 1803, 1804, 1808, 1810, 1864, 1903, 1911, 2091, 2118, and 2284 and the conjugate has less binding to FVIII inhibitory antibodies than the unconjugated polypeptide. In a further embodiment, the bioprotective polymer may be covalently attached to the polypeptide at one or more of the FVIII amino acid positions 81, 129, 377, 378, 468, 487, 491, 504, 556, 570, 711, 1648, 1795, 1796, 1803, 1804, 1808, 1810, 1864, 1903, 1911, 2091, 2118, and 2284, and at one or more of positions 377, 378, 468, 491, 504, 556, and 711 and the conjugate has less degradation of activity from a plasma protease capable of FVIII degradation than does the unconjugated polypeptide. Tin one embodiment, the plasma protease may be activated protein C.

In a further embodiment, the linker having a bioprotective moiety may be covalently attached to B-domain deleted FVIII at amino acid position 129, 491, 1804, and/or 1808. In a further embodiment, the linker having a bioprotective moiety may be attached to the polypeptide at FVIII amino acid position 1804 and comprises polyethylene glycol. As an example, the one or more predefined sites for linker attachment may be created by site specific cysteine mutation of BDD.

One or more sites on the functional FVIII polypeptide may be the predefined sites for linker attachment. In one embodiment, the polypeptide may have one linker attached. The linker can be multi-PEGylated, for example, mono-PEGylated or di-PEGylated.

The invention also relates to a method for the preparation of the FVIII-linker-PEG conjugate comprising mutating a nucleotide sequence that encodes for the functional FVIII moiety to substitute a cysteine residue at a pre-defined site in the encoded FVIII moiety; expressing the mutated nucleotide sequence to produce a cysteine-substituted mutein; purifying the mutein if required; reacting the mutein with the biocompatible polymer that has been activated to react with polypeptides at reduced cysteine residues, thereby forming a conjugate; and optionally purifying the conjugate. In another embodiment, the invention provides a method for site-directed PEGylation of a FVIII mutein comprising: (a) expressing a site-directed FVIII mutein wherein the mutein has a cysteine replacement for an amino acid residue on the exposed surface of the FVIII mutein and that cysteine is capped; (b) contacting the cysteine mutein with a reductant under conditions to mildly reduce the cysteine mutein and to release the cap; (c) removing the cap and the reductant from the cysteine mutein; and (d) after the removal of the reductant, treating the cysteine mutein with PEG comprising a sulfhydryl coupling moiety under conditions such that PEGylated FVIII mutein is produced. The sulfhydryl coupling moiety of the PEG is selected from the group consisting of thiol, triflate, tresylate, aziridine, oxirane, S-pyridyl and maleimide moieties.

In one embodiment, the invention relates to biosynthesis of the recombinant procoagulant factor in cell culture. The cell culture medium contains cysteines that “cap” the cysteine residues on the mutein by forming disulfide bonds. In the preparation of the conjugate, the cysteine mutein produced in the recombinant system is capped with a cysteine from the medium and this cap is removed by mild reduction that releases the cap before adding the cysteine-specific reagent. Other methods known in the art for site-specific mutation of FVIII may also be used, as would be apparent to one of skill in the art.

### Pharmaceutical Compositions

Based on well known assays used to determine the efficacy for treatment of conditions identified above in mammals, and by comparison of these results with the results of known medicaments that are used to treat these conditions, the effective dosage of the polypeptides of this invention may readily be determined for treatment of each desired indication. The amount of the active ingredient to be administered in the treatment of one of these conditions can vary widely according to such considerations as the particular polypeptide and dosage unit employed, the mode of administration, the period of treatment, the age and sex of the patient treated, and the nature and extent of the condition treated.

The application provides, in part, compositions comprising procoagulant factor conjugates as described herein. The compositions may be suitable for in vivo administration and are pyrogen free. The compositions may also comprise a pharmaceutically acceptable carrier. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Supplementary active ingredients also may be incorporated into the compositions.

The compositions of the present invention include classic pharmaceutical preparations. Administration of these compositions according to the present invention may be via any common route. The pharmaceutical compositions may be introduced into the subject by any conventional method, for example, by intravenous, intradermal, intramuscular, subcutaneous, intraperitoneal, or transdermal delivery, or by surgical implantation at a particular site. The treatment may consist of a single dose or a plurality of doses over a period of time.

The compositions of the invention can be lyophilized for storage and reconstituted into liquid for administration. Determination of a suitable carrier for formulation of the protein conjugate is within the skill in the art. One suitable lyophilization composition consists essentially of: the FVIII conjugate, 20 mM MOPS pH 6.8, 220 mM NaCl, 2.5 mM CaCl₂, 100 ppm Tween™ 80, and 1% sucrose. Formulations of the compositions advantageously do not contain serum components or proteins from any animal source. Recombinant protein, such as serum albumin, however, may be added to enhance stability. Additional stabilizing agents known in the art can be used in the formulation, including but not limited to glycine and/or sucrose.

The pharmaceutical forms, suitable for injectable use, include sterile aqueous solutions or dispersions and sterile powders for the extemporaneous preparation of sterile injectable solutions or dispersions. The form should be sterile and should be fluid to the extent that easy syringability exists. It should be stable under the conditions of manufacture and storage and should be preserved against the contaminating action of microorganisms, such as bacteria and fungi. The carrier may be a solvent or dispersion medium containing, for example, water, ethanol, polyol (e.g., glycerol, propylene glycol, and liquid polyethylene glycol, and the like) sucrose, L-histidine, polysorbate 80, or suitable mixtures thereof, and vegetable oils. The proper fluidity may be maintained, for example, by the use of a coating, such as lecithin, by the maintenance of the required particle size in the case of dispersion, and by the use of surfactants. The prevention of the action of microorganisms may be brought about by various antibacterial an antifungal agents, for example, parabens, chlorobutanol, phenol, sorbic acid, thimerosal, and the like. The injectable compositions may include isotonic agents, for example, sugars or sodium chloride. Prolonged absorption of the injectable compositions may be brought about by the use in the compositions of agents delaying absorption, for example, aluminum monostearate and gelatin.

Sterile injectable solutions may be prepared by incorporating the active compounds (e.g., procoagulant factor conjugates) in the required amount in the appropriate solvent with various of the other ingredients enumerated above, as required, followed by filtered sterilization.

Upon formulation, solutions may be administered in a manner compatible with the dosage formulation and in such amount as is therapeutically effective. “Therapeutically effective amount” is used herein to refer to the amount of a polypeptide that is needed to provide a desired level of the polypeptide in the bloodstream or in the target tissue. The precise amount will depend upon numerous factors, for example, the particular procoagulant factor conjugate, the components and physical characteristics of the therapeutic composition, intended patient population, mode of delivery, individual patient considerations, and the like, and can readily be determined by one skilled in the art, based upon the information provided herein.

The formulations may be easily administered in a variety of dosage forms, such as injectable solutions, and the like. For parenteral administration in an aqueous solution, for example, the solution should be suitably buffered, if necessary, and the liquid diluent first rendered isotonic with sufficient saline or glucose. These particular aqueous solutions are especially suitable for intravenous, intramuscular, subcutaneous and intraperitoneal administration.

The frequency of dosing will depend on the pharmacokinetic parameters of the agents and the routes of administration. The optimal pharmaceutical formulation may be determined by one of skill in the art depending on the route of administration and the desired dosage (see, e.g., Remington's Pharmaceutical Sciences, Mack Publishing Co., Easton, Pa., 20^{th} edition, 2000, incorporated herein by reference). Such formulations may influence the physical state, stability, rate of in vivo release, and rate of in vivo clearance of the administered agents. Depending on the route of administration, a suitable dose may be calculated according to body weight, body surface area, or organ size. Further refinement of the calculations necessary to determine the appropriate treatment dose is routinely made by those of ordinary skill in the art without undue experimentation, especially in light of the dosage information and assays disclosed herein, as well as the pharmacokinetic data observed in animals or human clinical trials. Exemplary dosing schedules include, without limitation, administration five times a day, four times a day, three times a day, twice daily, once daily, three times weekly, twice weekly, once weekly, twice monthly, once monthly, and any combination thereof.

Appropriate dosages may be ascertained through the use of established assays for determining blood clotting levels in conjunction with relevant dose response data. The final dosage regimen may be determined by the attending physician, considering factors that modify the action of drugs, for example, the drug's specific activity, severity of the damage, and the responsiveness of the patient, the age, condition, body weight, sex and diet of the patient, the severity of any infection, time of administration, and other clinical factors.

### Exemplary Uses

The compositions described herein may be used to treat a deficiency of a coagulation factor. In one embodiment, the bleeding disorder may be hemophilia. Symptoms of such bleeding disorders include, for example, severe epistaxis, oral mucosal bleeding, hemarthrosis, hematoma, persistent hematuria, gastrointestinal bleeding, retroperitoneal bleeding, tongue/retropharyngeal bleeding, intracranial bleeding, and trauma-associated bleeding. The average doses administered intraveneously are in the range of 40 units per kilogram for pre-operative indications, 15 to 20 units per kilogram for minor hemorrhaging, and 20 to 40 units per kilogram administered over an 8-hours period for a maintenance dose.

The compositions of the present invention may be used for prophylactic applications. In some embodiments, procoagulant factor conjugates may be administered to a subject susceptible to or otherwise at risk of a disease state or injury to enhance the subject's own coagulative capability. Such an amount may be defined to be a “prophylactically effective dose.” Administration of the procoagulant factor conjugates for prophylaxis includes situations where a patient suffering from hemophilia is about to undergo surgery and the polypeptide is administered between one to four hours prior to surgery. In addition, the polypeptides are suited for use as a prophylactic against uncontrolled bleeding, optionally in patients not suffering from hemophilia. Thus, for example, the polypeptide may be administered to a patient at risk for uncontrolled bleeding prior to surgery.

The conjugates, materials, compositions, and methods described herein are intended to be representative examples of the invention, and it will be understood that the scope of the invention is not limited by the scope of the examples. Those skilled in the art will recognize that the invention may be practiced with variations on the disclosed polypeptides, materials, compositions and methods, and such variations are regarded as within the ambit of the invention.

The following examples are presented to illustrate the invention described herein, but should not be construed as limiting the scope of the invention in any way.

### EXAMPLES

In order that this invention may be better understood, the following examples are set forth. These examples are for the purpose of illustration only, and are not to be construed as limiting the scope of the invention in any manner. All publications mentioned herein are incorporated by reference in their entirety.

### Example 1

### Conjugation of PEG to a rFVIII Mutein

Peptides with a maleimide moiety at the C-terminus were commercially synthesized by BioPeptide. Some peptides were prepared having a short-chain PEG spacer (e.g., 4-unit PEG, “PEG4” and 12-unit PEG, “PEG12”). Short-chain PEG spacers are small, for example, less than about 2 kD. These PEG units are in addition to a large branched or unbranched bioprotective PEG moiety that is subsequently attached at, for example, the amino terminus of the peptide.

TABLE 1

| Linker | | |
|---|---|---|
| identifier | Prolinker Structure | SEQ ID NO. |
| A | DHHHHHHQGRGLK-maleimide | SEQ ID NO. 9 |
| B | GGGLVPRGSGK-maleimide | SEQ ID NO. 10 |
| C | GGGLTRIVGLVPRGSGK-maleimide | SEQ ID NO. 11 |
| D | GGGLTRIVGLVPRGSGK-PEG4-maleimide | SEQ ID NO. 12 |
| E | GGGLTRIVGLVPRGSGK-PEG12-maleimide | SEQ ID NO. 13 |
| F | nTPRSNRGK-PEG4-maleimide | SEQ ID NO. 14 |
| G | nTPRSNRGK-PEG12-maleimide | SEQ ID NO. 15 |
| H | LTPRRNRGK-PEG4-maleimide | SEQ ID NO. 16 |
| I | LTPRRNRGK-PEG12-maleimide | SEQ ID NO. 17 |
| J | GGGLTRIVGLVPRGSGKGGGLTRIVGLVPRGSGK- | SEQ ID NO. 18 |
| | maleimide | |

“n” is norleucine and the italicized letters are not amino acids.

Peptide-PEG and protein purifications were performed on a Pharmacia AKTA prime system. Pre-packed Superdex-200 column (10/300GL) and Sephadex G-75 resin were from Pharmacia. Monoclonal antibody-linked beads were prepared by standard methods. The antibody was directed against human FVIII C7F7.

Chromogenic assays of PEGylated FVIII were performed using Coatest SP FVIII kit from Chromogenix. Calibrated Automated Thrombogram (CAT) assays were performed on a Hemker Thrombinoscope BV instrument. The aPTT assays were performed on an Electra 1800C Automatic Coagulation Analyzer.

PEG-linkers were synthesized by forming a Schiff base between PEG-butyraldehyde (Nektar) and the N-terminal amine of the peptides described in Table 1. The Schiff base was then reduced to the corresponding amine by treatment with sodium cyanoborohydride. Typically, peptide (in 3-fold excess) was mixed with PEG-butyraldehyde in NaCNBH₃-containing coupling solution (Sigma, NaCNBH₃ in large excess) and stirred at room temperature for two hours before the reaction mixture was directly applied to a self-packed Sephadex G-75 column. Purification of the PEG-linker was achieved by eluting the column with deionized water.

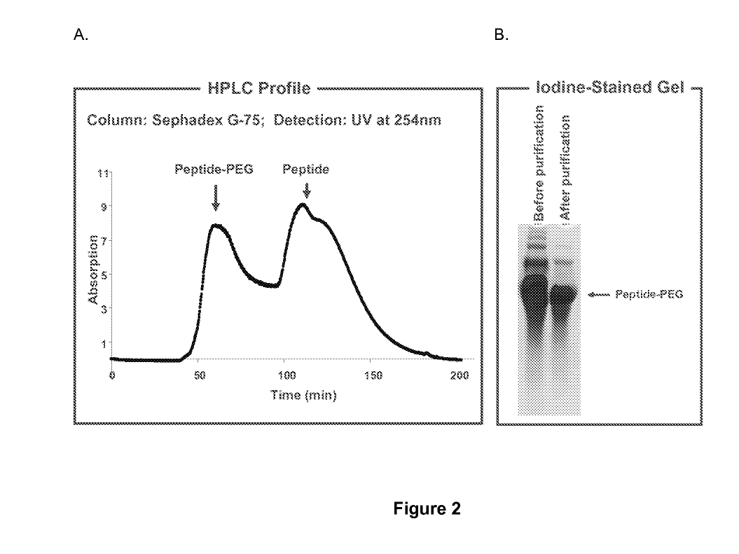

FIG. 2 shows an HPLC purification profile (A) and iodine gel-staining (B) of the PEG-linker before and after purification. The PEG-linker has a much higher molecular weight (30 kD) compared to the prolinker peptide (about 2 to about 3 kD).

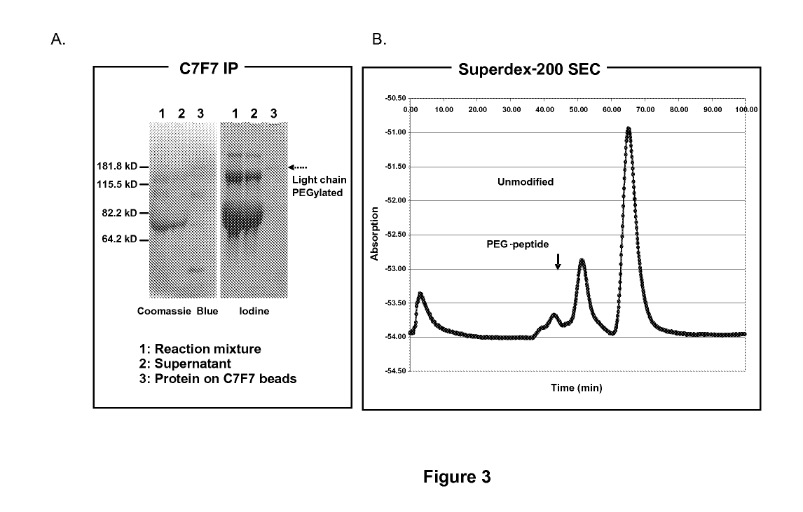

The PEG-linkers were then linked to a FVIII mutein in the presence of TCEP. Briefly, TCEP (final concentration 1 mM) was added to the FVIII mutein in formulation buffer (20 mM MOPS pH 6.8, 220 mM NaCl, 2.5 mM CaCl₂, 100 ppm Tween™ 80, and 1% sucrose) and the mixture incubated at 4° C. for 2 hours before PEG-linker was introduced at a final concentration of 100-200 μM. The mixture was incubated at 4° C. overnight (with gentle stirring) before the PEG-conjugated protein was purified by using C7F7 beads. After adhering the conjugate to the beads, the beads were washed with washing solution (20 mM MOPS pH 6.8, 220 mM NaCl, 2.5 mM CaCl₂, 100 ppm Tween™ 80) to remove other components. The product was eluted with eluting solution: 500 mM NaCl, 1 mM CaCl₂, 20 mM imidazole pH 7.0, and 100 ppm Tween™ 80. The product was added to a Superdex-200 column and eluted with formulation buffer lacking sucrose. FIG. 3 shows a typical result after C7F7 immunoprecipitation and Superdex-200 purification (HPLC profile). In FIG. 3, the linker is C from Table 1.

### Example 2

### Removal of PEG from PEG-conjugated FVIII by Thrombin

The PEG-linker-FVIII was readily removed from FVIII in the presence of thrombin where the linker had a thrombin recognition and cleavage site.

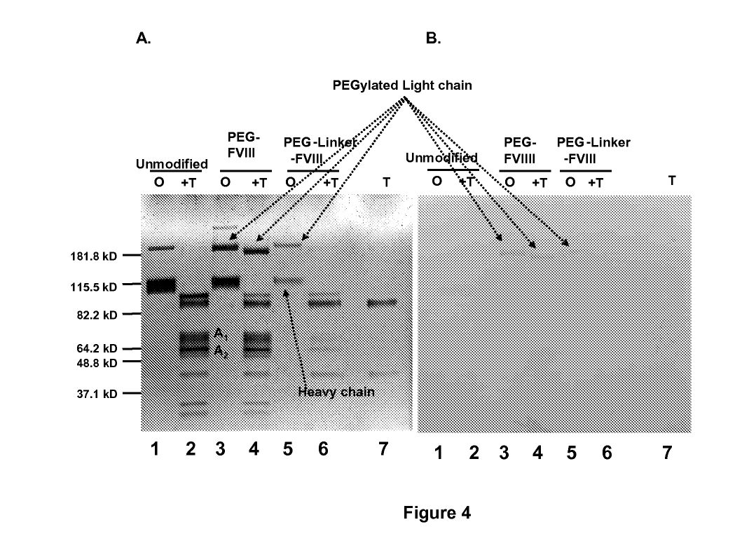

FIG. 4 illustrates separation of the FVIII mutein by SDS-polyacrylamide gel electrophoresis stained for protein (top left panel), SDS-PAGE stained with iodine for PEG (top right panel), and in diagrammatic form (bottom panel). The intact and thrombin-digested patterns of unmodified FVIII mutein are shown in lanes 1 and 2, respectively. The FVIII (about 2 units) was mixed with thrombin (0.2 units) in 20 μl formulation buffer and digested for 30 min at 37° C. After thrombin digestion, the FVIII light and heavy chains disappear and are replaced by cleavage products. Lane 7 has thrombin only. Polypeptides in lanes 1, 2, or 7 do not show the presence of any PEG. FVIII mutein directly (i.e., non-cleavably) conjugated to PEG (PEG-FVIII) is shown in lane 3. Upon treatment with thrombin of the FVIII with non-cleavable PEG, there was no removal of PEG-conjugated light chain (lane 4). After the PEG-linker-FVIII (about 2 units) was mixed with thrombin (0.2 unit) in 20 μl formulation buffer and digested for 30 min at 37° C., the PEG-conjugated light chain almost completely disappeared in SDS-PAGE analysis, indicating a complete removal of PEG (compare lanes 5 and 6). Thus, thrombin cleaves at the A1-A2 activation site, the A3 activation site, and the linker cleavage site.

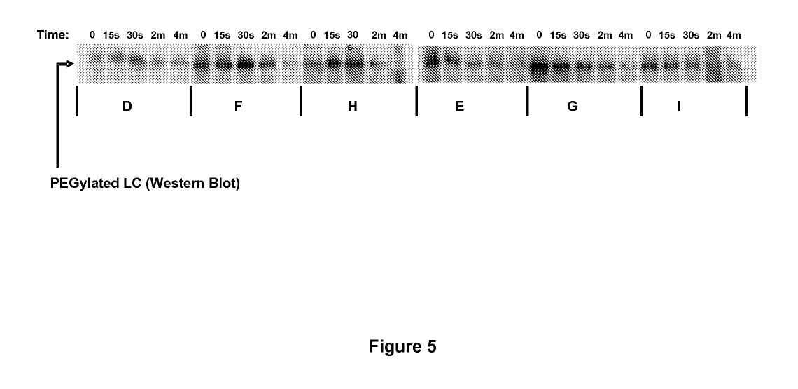

FIG. 5 depicts the time course of cleavage of FVIII conjugates shown as Western blots of FVIII light chain comprising the D, E, F, G, H, and I linkers according to Table 1.

### Example 3

### Activity of Cleavable-PEG Modified FVIII

Three bioassays relating to different stages of the coagulation pathway were performed on the PEG-cleavable linker-FVIII, to compare with the non-cleavable PEG-modified FVIII: 1) Chromogenic assay which detects the generation of FXa; 2) Thrombin generation assay which measures rate of thrombin generation (“CAT); and aPTT assay which measures speed of plasma coagulation. The results are listed in Tables 2 (PEG-FVIII) and 3 (PEG-Linker-FVIII).

TABLE 2

| | | PEG-FVIII | | |
|---|---|---|---|---|
| | FVIII | (non-cleavable) | PEG-peptide C-FVIII | |
| CAT | ~6 U/μg | 2.0 U/ml | 54 | U/ml |
| Chromogenic | ~6 U/μg | 2.0 U/ml | (52 ± 7) | U/ml |
| aPTT | ~6 U/μg | 0.27 U/ml | (5.1 ± 0.2) | U/ml |

| | | | |
|---|---|---|---|
| Chromogenic/aPTT | 1.0 | 7.4 | 10.2 ± 1.8 |
| Ratio | | | |

TABLE 3

| | | PEG- | PEG- | PEG- | PEG- | PEG- | PEG- |
|---|---|---|---|---|---|---|---|
| | PEG- | linker D- | linker F- | linker H- | linker E- | linker G- | linker I- |
| | FVIII | FVIII | FVIII | FVIII | FVIII | FVIII | FVIII |
| Chromogenic | 2.0 U/ml | 2.5 U/ml | 2.0 U/ml | 1.9 U/ml | 2.4 U/ml | 2.0 U/ml | 2.3 U/ml |
| aPTT | 0.27 U/ml | 0.19 U/ml | 0.17 U/ml | 0.09 U/ml | 0.2 U/ml | 0.15 U/ml | 0.13 U/ml |
| Chromogenic/ | 7.4 | 13 | 12 | 21 | 12 | 13 | 18 |
| aPTT | | | | | | | |

### Example 4

### Measurement of Pharmacokinetics in the Hemophilic Mouse

Hemophilic C57/BL6 mice having a disrupted FVIII gene are injected i.v. with about 2.5 IU FVIII (control) or PEG-linker-FVIII samples in a volume of about 0.1 ml. At various time points, mice are bled for measurement of FVIII activity using the chromogenic activity to determine the circulation half-life activity of the control and test samples.

### Example 5

### Efficacy of Procoagulant Conjugates in a Laceration Model

Hemophilic C57/BL6 mice are weighed and anesthetized. The inferior vena cava is exposed and 0.1 ml of saline (control) or a PEG-linker-FVIII sample (˜2.5 IU) is injected. Pressure is applied to the injection site to minimize bleeding. After two minutes, the right kidney is exposed and lacerated to a uniform depth. Blood loss is measured as a function of dose and type.

On the basis of the above disclosure, one of skill in the art can formulate the procoagulant conjugates of the invention for the treatment of coagulation disorders (e.g., hemophilia). The conjugates will be cleaved or substantially cleaved in situ to release procoagulant or coagulant to catalyze blood clotting, essentially by providing a procoagulant (e.g., FVIII) at the site where procoagulant activity is required.

All publications and patents mentioned in the above specification are incorporated herein by reference. Various modifications and variations of the described methods of the invention will be apparent to those skilled in the art without departing from the scope and spirit of the invention.

Although the invention has been described in connection with specific embodiments, it should be understood that the invention as claimed should not be unduly limited to such specific embodiments. Indeed, various modifications of the above-described modes for carrying out the invention which are obvious to those skilled in the field of biochemistry or related fields are intended to be within the scope of the following claims. Those skilled in the art will recognize, or be able to ascertain using no more than routine experimentation, many equivalents to the specific embodiments of the invention described herein. Such equivalents are intended to be encompassed by the following claims.

1. A conjugate comprising an endopeptidase-activatable procoagulant factor and one or more bioprotective moieties wherein the procoagulant factor is linked to the bioprotective moiety by a linker which comprises one or more cleavage sites recognized by the endopeptidase, such that the bioprotective moiety is released from the procoagulant factor in the presence of the endopeptidase.

2. The conjugate of claim 1, wherein the procoagulant factor is selected from the group consisting of a FV moiety, a FVII moiety, a FVIII moiety, a FIX moiety, a FX moiety, and a thrombin moiety.

3. The conjugate of claim 1, wherein the procoagulant factor is selected from the group consisting of a FVII moiety, a FVIII moiety, and a Factor IX moiety.

4. The conjugate of claim 3 wherein the procoagulant factor is a recombinant FVIII moiety or Factor IX moiety.

5. The conjugate of claim 1, wherein the bioprotective moiety is selected from the group consisting of a hydrophilic polymer, polysaccharide, polysialic acid, albumin, immunoglobulin, and fragment of immunoglobulin.

6. The conjugate of claim 5, wherein the hydrophilic polymer is polyethylene glycol (PEG).

7. The conjugate of claim 6, wherein said PEG comprises PEG having a molecular weight of between about 10 kD and about 300 kD.

8. The conjugate of claim 7, wherein said PEG is linear.

9. The conjugate of claim 5, wherein the polysaccharide is a starch.

10. The conjugate of claim 9, wherein the starch is selected from hydroxyethyl-starches and hydroxy propyl-starches.

11. The conjugate of claim 1, wherein the linker comprises a thrombin or Factor Xa cleavage site.

12. The conjugate of claim 11, wherein the linker comprises a thrombin cleavage site and the endopeptidase is thrombin.

13. The conjugate of claim 12, wherein the cleavage site is selected from the group consisting of SEQ ID NO. 1 and SEQ ID NO. 2.

14. The conjugate of claim 11, wherein the linker comprises a Factor Xa cleavage site and the endopeptidase is Factor Xa.

15. The conjugate of claim 14, wherein the cleavage site is selected from the group consisting of SEQ ID NO. 3, SEQ ID NO. 4, and SEQ ID NO. 5.

16. The conjugate of claim 14, wherein the cleavage site is selected from the group consisting of SEQ ID NO. 6, SEQ ID NO. 7, and SEQ ID NO. 8.

17. The conjugate of claim 6, wherein said PEG is attached to an amino-terminus amino acid residue of said linker.

18. The conjugate of claim 1, wherein the linker further comprises a spacer between the cleavage site and the procoagulant factor.

19. The conjugate of claim 1, wherein the linker is attached to a naturally-occurring or introduced cysteine residue in said protein.

20. The conjugate of claim 1, wherein the linker comprises SEQ ID NO. 9, SEQ ID NO. 10, SEQ ID NO. 11, SEQ ID NO. 12, SEQ ID NO. 13, SEQ ID NO. 14, SEQ ID NO. 15, SEQ ID NO. 16, or SEQ ID NO. 17.

21. The conjugate of claim 1, wherein the linker is attached to an amino acid other than a terminal amino acid residue of the protein.

22. A therapeutic composition comprising the conjugate of any one of claims 1-22.

23. A method of treating procoagulation factor deficiency in a subject, which comprises administering to the subject a therapeutically-effective amount of an endopeptidase-activatable conjugate, which conjugate comprises a procoagulation factor moiety conjugated to one or more bioprotective moieties by means of a linker, wherein the linker provides one or more cleavage sites which is recognized by an endopeptidase, whereby the bioprotective polymer moiety is cleaved from the procoagulation factor in vivo to provide substantially unconjugated procoagulation factor moiety in the subject.

24. The method of claim 23 wherein the endopeptidase is thrombin and the cleavage site is a cleavage site recognized by thrombin.

25. The method of claim 23, wherein the procoagulation factor is selected from FV, FVII, FVIII, FIX, FX, and thrombin.

26. The method of claim 25, wherein the procoagulation factor is FVIII.

27. A FVIII conjugate which is reactive in vivo to provide active FVIII moiety at an in vivo microlocus of proteolytic conversion of fibrinogen to fibrin, which conjugate comprises a FVIII moiety and a bioprotective moiety, which conjugate is cleaved in vivo at the site of proteolytic conversion to provide substantially unconjugated, active FVIII moiety at the site of proteolytic conversion.


SEQUENCE LISTING (18 sequences)

SEQ ID NO: 1 - protein, 6 aa, Artificial Sequence
  Linker Sequence
XXPRXX

SEQ ID NO: 2 - protein, 4 aa, Artificial Sequence
  Linker Sequence
GRXX

SEQ ID NO: 3 - protein, 5 aa, Artificial Sequence
  Linker sequence
IEGRX

SEQ ID NO: 4 - protein, 5 aa, Artificial Sequence
  Linker sequence
IDGRX

SEQ ID NO: 5 - protein, 5 aa, Artificial Sequence
  Linker sequence
AEGRX

SEQ ID NO: 6 - protein, 5 aa, Artificial Sequence
  Linker sequence
IEGRX

SEQ ID NO: 7 - protein, 5 aa, Artificial Sequence
  Linker sequence
IDGRX

SEQ ID NO: 8 - protein, 5 aa, Artificial Sequence
  Linker sequence
AEGRX

SEQ ID NO: 9 - protein, 13 aa, Artificial Sequence
  Linker sequence
DHHHHHHQGRGLK

SEQ ID NO: 10 - protein, 11 aa, Artificial Sequence
  Linker sequence
GGGLVPRGSGK

SEQ ID NO: 11 - protein, 17 aa, Artificial Sequence
  Linker sequence
GGGLTRIVGLVPRGSGK

SEQ ID NO: 12 - protein, 17 aa, Artificial Sequence
  Linker sequence
GGGLTRIVGLVPRGSGK

SEQ ID NO: 13 - protein, 17 aa, Artificial Sequence
  Linker sequence
GGGLTRIVGLVPRGSGK

SEQ ID NO: 14 - protein, 9 aa, Artificial Sequence
  Linker sequence
XTPRSNRGK

SEQ ID NO: 15 - protein, 9 aa, Artificial Sequence
  Linker sequence
XTPRSNRGK

SEQ ID NO: 16 - protein, 9 aa, Artificial Sequence
  Linker sequence
LTPRRNRGK

SEQ ID NO: 17 - protein, 9 aa, Artificial Sequence
  Linker sequence
LTPRRNRGK

SEQ ID NO: 18 - protein, 34 aa, Artificial Sequence
  Linker sequence
GGGLTRIVGLVPRGSGKGGGLTRIVGLVPRGSGK